(19) United States
(12) Patent Application Publication
(10) Pub. No.: US 2006/0025799 A1
(43) Pub. Date: Feb. 2, 2006

(54) Endoscopically placed gastric balloon (EPGB) device and method for treating obesity involving the same

(76) Inventor: Patrick Basu, Pelham Manor, NY (US)

Correspondence Address: LACKENBACH SIEGEL, LLP, LACKENBACH SIEGEL BUILDING, 1 CHASE ROAD, SCARSDALE, NY, 10583, US

(21) Appl. No.: 11/189,389

(22) Filed: Jul. 26, 2005

Related U.S. Application Data
(60) Provisional application No. 60/591,350, filed on Jul. 27, 2004.

(51) Int. Cl. A61M 29/00

(52) U.S. Cl. 606/192

(57) ABSTRACT

An endoscopically placed gastric balloon (EPGB) includes a gastric balloon. The EPGB is insertable in a patient and anchored in position between the lock and a portal. A port section enables the gastric balloon to be filled with mass containing a liquid medium during treatment, resulting in a patient receiving a true satiated neurogastric full feeling eliminating cravings and hunger feelings. The EPGB device allows for the balloon to be easily deflated or reduced and for a gastric suction and lavage to be accomplished without patient discomfort, while the device is in place and easily concealed from others during use. A method of using the EPGB enables a clinical treatment of a morbidly obese patient with greatly reduced health risks and neuro-psychiatric complications.

### RELATED APPLICATIONS

This application claims priority from U.S. Provisional Patent Application No. 60/591,350 filed Jul. 27, 2005, the entire contents of which are herein incorporated by reference.

The contents of Disclosure Documents No. ______ (filed May 14, 2003) entitled Endoscopically Placed Gastric Balloon (EPGB) for Morbid Obesity, and Disclosure Document No. ______ (filed May 19, 2003) entitled Endoscopically Placed Gastric Balloon (EPGB) for Morbid Obesity and Device, are referred to and specifically incorporated herein by reference in their entireties. A timely request is made to retain these documents within the file.

### FIGURE SELECTED FOR PUBLICATION

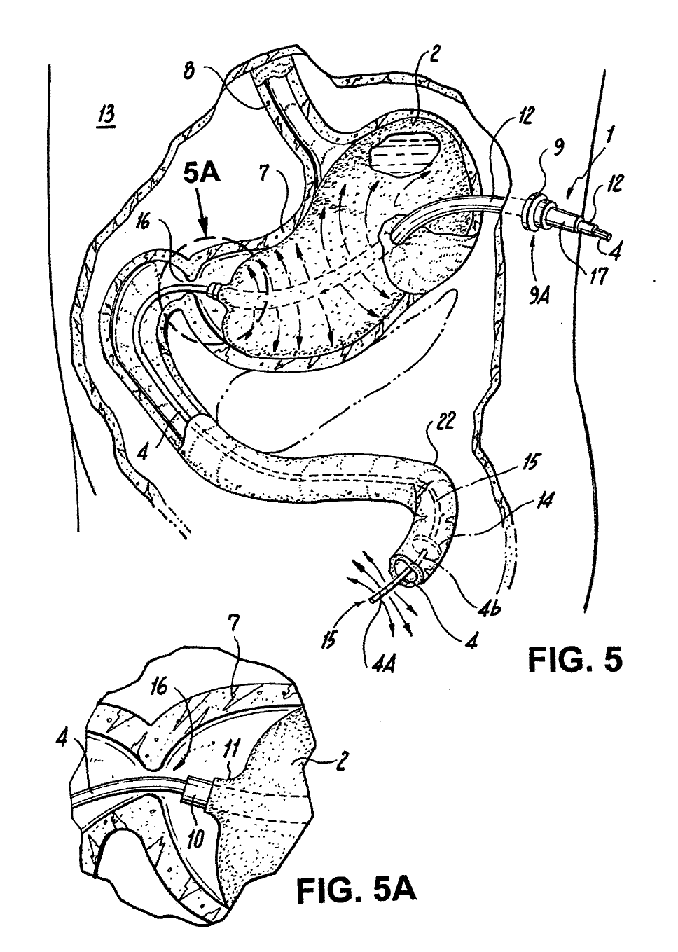

Applicant selects FIG. 5 for future purpose of publication.

### BACKGROUND OF THE INVENTION

1. Field of the Invention

The present invention relates an Endoscopically Placed Gastric Balloon (EPGB) device and a method of treating morbid obesity using the same. More specifically, the present invention relates to a reversibly implantable medical device, causing a patient, when employed (inflated/filled), to feel satiated while allowing for enteral feeding and gastric cleaning during treatment, and a method for treating morbid obesity employing the device.

2. Description of the Related Art

Over the last several decades, beginning in the 1940's-1950's, uncontrolled weight gain and obesity has become a clinical and/or endemic problem globally, particularly in certain countries such as the United States of America. Obesity has multiple causes or spectrums of causes including generic, constitutional, hormonal, and general lack of energy expenditure, which in turn can have profound effect on entire families and even multi-generational levels within families.

The medical profession, hospitals, health associations and other health related organizations, have responded to this growing problem by developing and implementing various health newsletters, fitness guidelines, and fitness programs, as well as body weight measurement tools useful in gauging the impact of a patient's weight in addressing the problem of obesity on one's overall health.

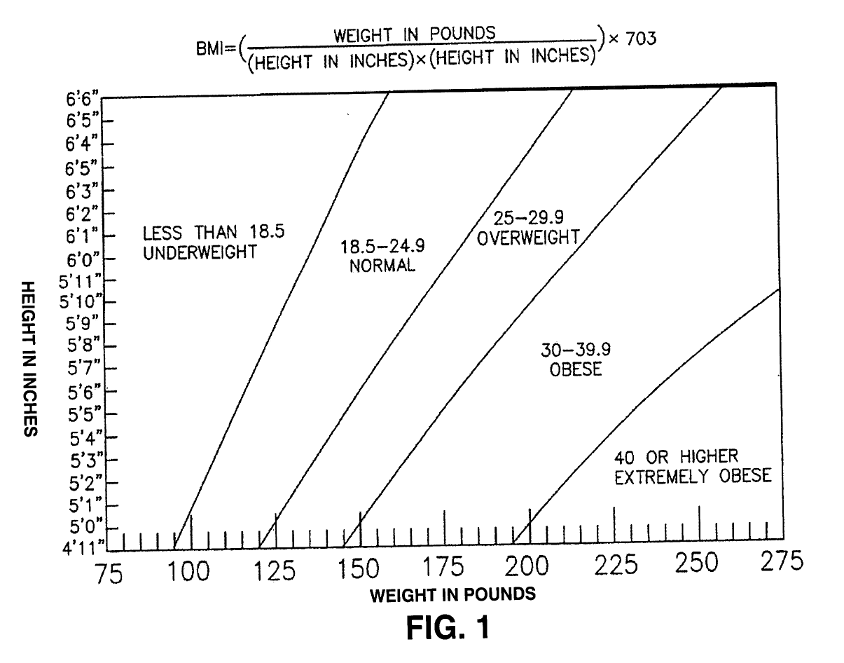

Referring now to FIG. 1, one of the measurement tools clinicians use is the body mass index (BMI) of a patient. A Body Mass Index (BMI) chart allows an easy graphical visualization of a patients body mass index for a patients respective height in inches and weight in pounds. Using the BMI chart, and understanding a patient's physical condition, a physician is better positioned in guiding a patient to improved health. In viewing the chart it is to be understood, that the ranges provided are provided as illustrative groupings only.

It is common knowledge that being overweight, obese, or morbidly obese has substantial morbidity on a patient's overall health, including impact on major body organs such as the heart and liver, and the risks posed to the development of cancer. In addition, having been diagnosed with cancer, a patient's odds of survival maybe effected by their weight depending on the type of cancer that a patient may develop. See the New England Journal of Medicine (N. Engl. J. Med 348;17) Apr. 24, 2002, pg. 1625-1638 and other related references.

Obesity has been defined as an increase in body mass index (BMI) of 30% or higher. Morbid obesity is normally defined in those patients having a BMI over 35% and who are at grave risk of experiencing fatal health consequences. It has been estimated that in two decades, morbid obesity will prevail in over 46% of the United States population. As an example of this problem, a recent study of teenagers in the New York City Public School System revealed that about 40% of those students were overweight.

There have been several designer-type weight loss programs and behavioral programs created in an effort to combat the potential crisis in obesity. These programs have been heavily advertized and are widely known. Many of these programs have resulted in significant weight loss-gain cycles, creating an undulating weight-loss/weight-gain phenomenon or yo-yo diet effect.

Recent bariatric surgical techniques have been employed to combat this type of undulating clinical syndrome. These bariatric approaches involve the use of stomach staples, or gastric bypass surgery to implement a surgical approach within operating hospitals to provide a bio- or neuro-behavioral affect generating a satiated feeling by influencing a patient's gastric inlet. The gastric inlet is known to provide a biometric feedback sensation to a patient's brain resulting in either a feeling of a full or empty stomach.

Unfortunately, many of the present surgical approaches with neuro-behavioral modifications, particularly the bariatric approach, still have significant morbidity. Such morbidity may lead to other health related problems.

There are several postulations about obesity from the constitutional, genetic, metabolic, hormonal and neuro-psychiatric dysfunctions all of which lead to this heterogeneous clinical syndrome called morbid obesity. Consequently, despite all the weight loss programs, exercise and biochemical manipulations with fat-burners, etc., obesity is still a perplexing and difficult to treat phenomenon.

As obesity eventually proceeds to severe morbidity, effecting quality of life and life style, and leading to often-fatal cardio-pulmonary dysfunction and hepatic cirrhosis, improved clinical solutions are needed that do not expose a patient to the complications and risks associated with stomach reduction and prevent the undulatory effect often associated with common diet regiments, weight-loss, and other behavioral programs.

Many solutions to weight gain and excess weight have been proposed and tested. See Mayo Clinic Special Report: Weight Control, Mayo Clinic Health Information, Mayo Foundation for Medical Education and Research, (pg. 1-8, MC2493-13/R0800), and Gastroenterology, (ISSN 0016-5085, Vol. 124, No. 4, April 2003) for a general discussion of cancer mortality in obesity subjects and a discussion of the medical effects of obesity.

Lately, in previous approaches to gastric balloon intervention, continuing via surgery was discontinued because of balloon ingestion or balloon migration causing embolization and an increased surgical risk. Consequently, the present clinical regulated-feeding programs have been largely unsuccessful to date.

As a consequence, there is a need for an improved method and apparatus for treating morbid obesity that responds to the concerns noted above.

### OBJECTS AND SUMMARY OF THE INVENTION

In response to the needs noted above, the present invention provides an endoscopically placed gastric balloon (EPGB) and a method for treating morbid obesity.

The present invention, in alternative embodiments, provides an EPGB and a method that simulates physically and physiologically a completely full and satiated feeling, thereby manipulating the neuro-hormonal and receptive links involved in the brain-gut-axis regarding obesity dysfunction.

The present invention, in alternative embodiments provides an EPGB device that is easily implanted using simple endoscopic surgical techniques and provides for easy manipulation during clinical observation and treatment.

The present invention, in alternative embodiments provides an easily implemented method for treating morbid obesity using an EPGB-suitable device in patients with morbid obesity.

The present invention also relates to an endoscopically placed gastric balloon (EPGB) that includes an inflatable gastric balloon. The EPGB is removably insertable in a patient and anchored in position between the lock and a stoma. A water port section enables the gastric balloon to be filled with fluid or alternatively a gas during treatment, resulting in a patient receiving a true satiated neuro-hormonal state, minimizing cravings and hunger. The EPGB device allows the balloon to be easily reduced and a gastric suction and lavage to be accomplished without patient discomfort, thereby minimizing gastric damage to the balloon. A method of using the EPGB enables a clinical treatment of a morbidly obese patient with greatly reduced health hazards and a consequential reduced risk of early demise.

According to an embodiment of the present invention there is provided an endoscopically placed gastric balloon (EPGB) device, comprising: a main tube having a defined central axis and at least a first end opposite a second end; at least a first liquid chamber sealed to an outer diameter of said main tube; at least one balloon sealing bounded at opposite ends of said liquid chamber proximate respective third and fourth sealed ends; means for communicating a fluid pressure between said liquid chamber and said balloon; and means for injecting a preselected fluid into said liquid chamber, through said means for communicating, and for distending said balloon during a use of said device.

According to another embodiment of the present invention, there is provided a method for treating morbid obesity, comprising the steps of: surgically positioning an endoscopically placed gastric balloon (EPGB) device in a suitable patient, said EPGB comprising: a main tube having a defined central axis and at least a first end opposite a second end; at least a first liquid chamber sealed to an outer diameter of said main tube; said liquid chamber sealed to said main tube at a third and a fourth sealed ends; at least one balloon sealing bounded at opposite ends of said liquid chamber proximate respective third and fourth sealed ends; means for communicating a fluid pressure between said liquid chamber and said balloon; and means for injecting a preselected fluid into said liquid chamber, through said means for communicating, and for distending said balloon during a use of said device, thereby causing a patient to feel a satiated hunger; inflating said device during said use, whereby said patient feels a satiated hunger craving; deflating and operating said device to remove, wash, and suction damaging fluids from said patient's stomach; and feeding said patient via during one of said steps of inflating and deflating, whereby the nutritional requirements of said patient are maintained in a convenient manner, while maintaining said satiated hunger craving status.

The above, and other objects, features and advantages of the present invention will become apparent from the following description read in conduction with the accompanying drawings, in which like reference numerals designate the same elements.

### BRIEF DESCRIPTION OF THE DRAWINGS

FIG. 1 is a standardized Body Mass Index (BMI) chart.

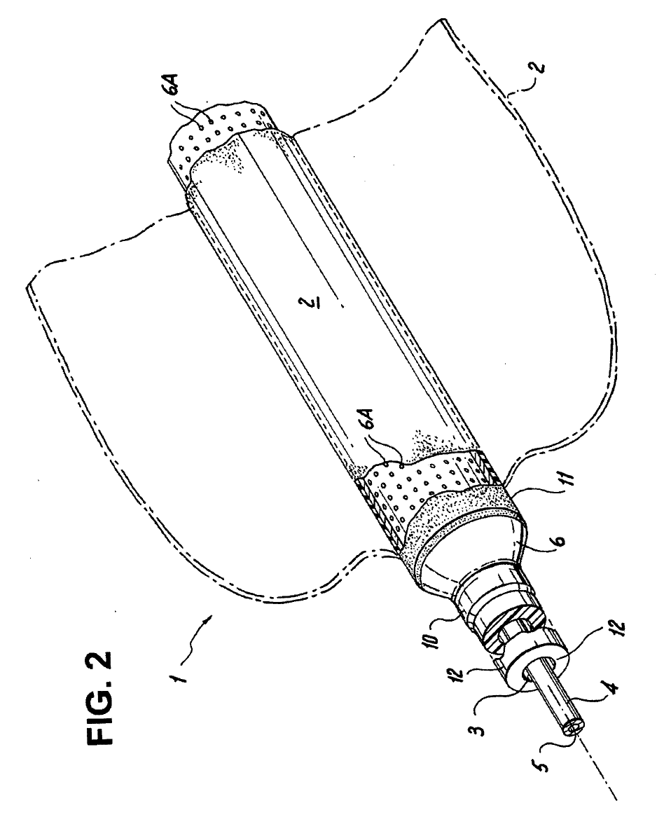

FIG. 2 is a diagrammatic view of a portion of one alternative embodiment of an endoscopically placed gastric balloon (EPGB) device for treating morbid obesity.

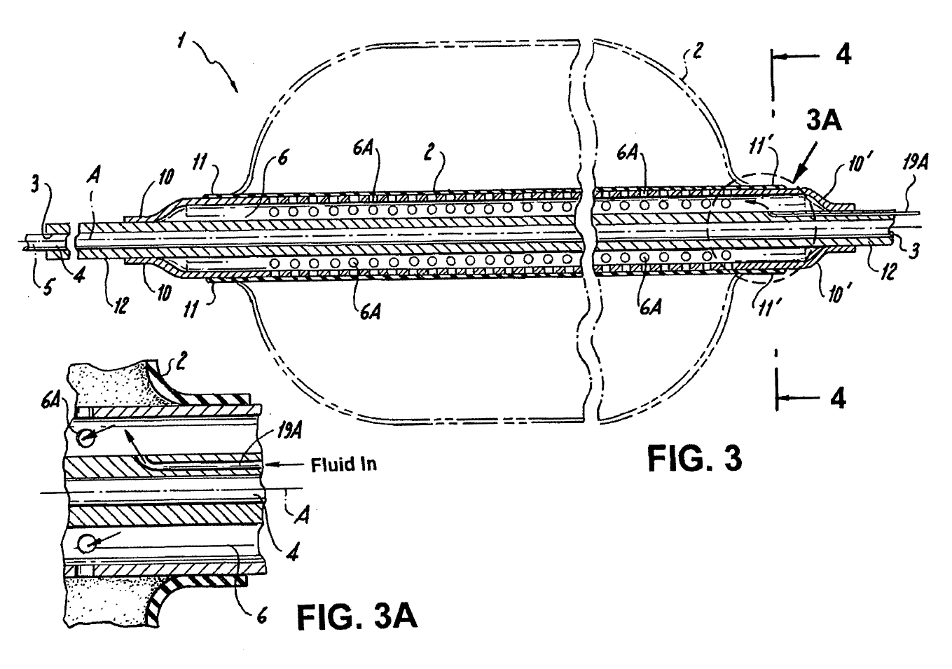

FIG. 3A is an enlarged cross section of the EPGB device shown in FIG. 3.

FIG. 3 is a partial side view of one alternative embodiment of EPGB device showing both an inflated and a deflated position.

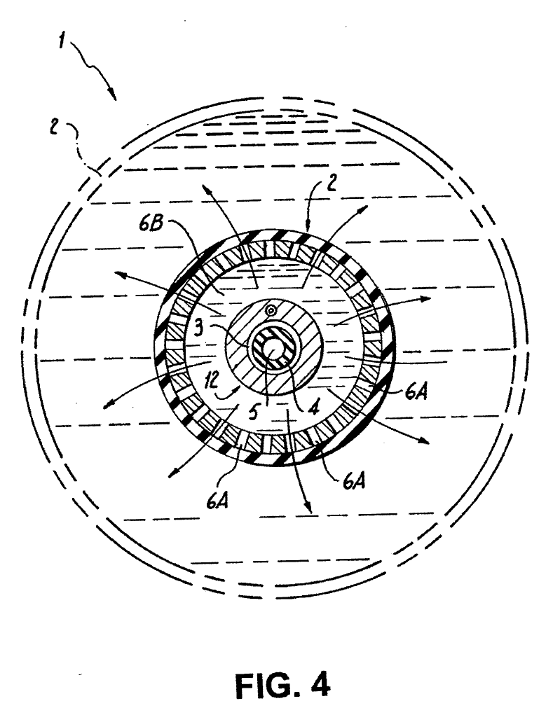

FIG. 4 is an axial view of one alternative embodiment of the EPGB device in a distended condition along section I-I of FIG. 2.

FIG. 5 is diagrammatic view of the EPGB device positioned within a patient with the balloon in an extended condition.

FIG. 5A is a partial view of a portion of the EPGB device exiting the stomach.

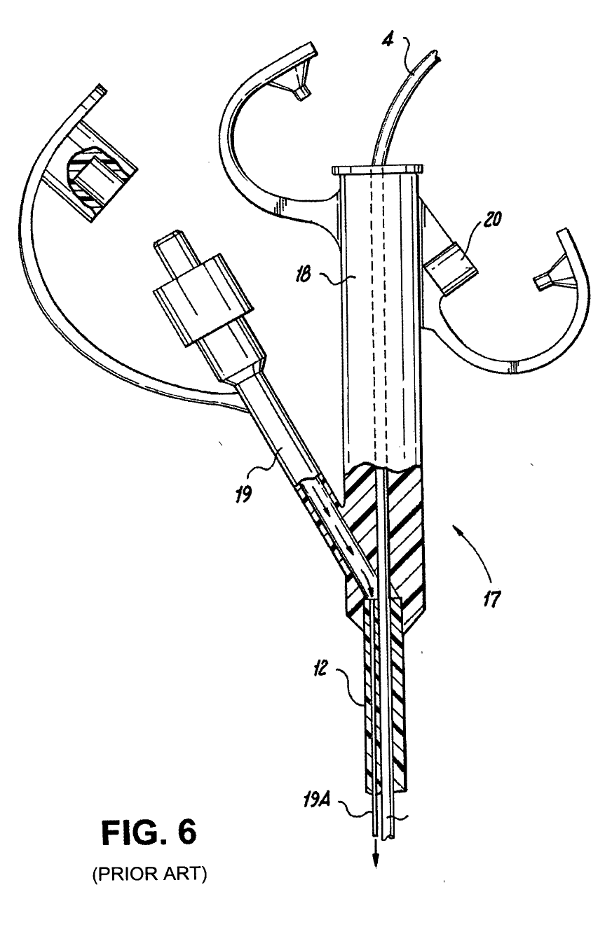

FIG. 6 is a partial side view of one adaptation of a conventional feeding tube end with multiple ports for incorporation with one alternative embodiment of an EPGB device.

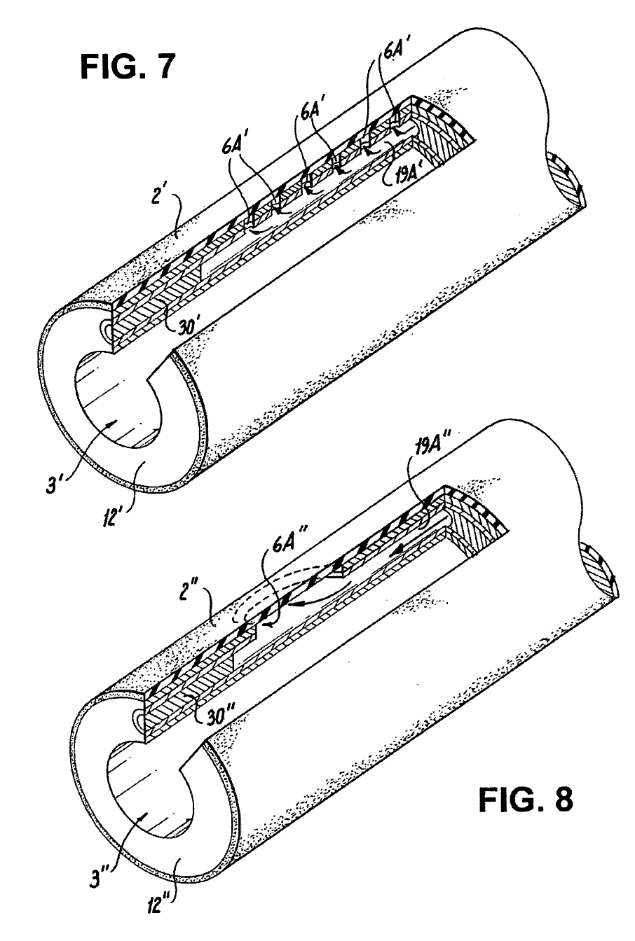

FIG. 7 is a partially cut-away view of another alternative embodiment of an end of the EPGB device.

FIG. 8 is a partially cut-away view of another embodiment of an end of the EPGB device.

### DETAILED DESCRIPTION OF THE PREFERRED EMBODIMENTS

I. Endoscopically Placed Gastric Balloon (EPGB) Device

Referring now to FIGS. 2 through 8, according to a first embodiment of the present invention, an endoscopically placed gastric balloon (EPGB) device 1 is shown. Generally, an EPGB device 1 includes a partially surrounding gastric balloon 2, as will be explained. In use, gastric balloon 2 is inflatable with a suitable fluid (for example, water) or a combination of a suitable fluid and a suitable gas (for example, air), and the contained and bounded fluid mass is affective in providing a beneficial full-stomach-feeling, so important when treating patients with neuro-psychiatric dysfunctions. According to the present invention, the mass, providing a downward pressure, and biometric and bio-psychiatric feed-back provided by a mass of non-consumed fluid in the stomach is very effective in treating patients with morbid obesity and allowing patients to interpret the sensation as a full-stomach feeling.

In use, gastric balloon 2 may extend in a non-symmetrical and non-uniform shape relative to main tube 12 depending upon the effects of gravity, a patient's stomach, the particular amount of liquid used, and whether a liquid and gas combination is used.

In the embodiment shown, a liquid chamber 6 extends from main tube 12 at opposing first and second sealed ends 10, 10′. Gastric balloon 2 elastically extends from a main tube 12 and liquid chamber 6 at opposing first and second sealed balloon ends 11, 11′, as shown. First and second sealed balloon ends 11, 11′ sealing join gastric balloon 2 to an outer diameter of liquid chamber 6 and main tube 12 in a leak-proof manner. First and second sealed ends 10, 10′ sealingly join liquid chamber 6 to an outer diameter of main tube 12 and prevent water escape. In this manner, the EPGB device 1 provides an integral and sealed unit, ensuring patient safety and operational reliability.

For ease of reference it should be understood that a central axis A, alternatively and interchangeably called a longitudinal axis A, extends generally and not precisely along the length of main tube 12, and particularly along the region including liquid chamber 6. Since main tube 12 is flexible, and in manufacture, use, or storage, may flex, it should be understood, that central axis/longitudinal axis A is the central axis as continually defined through selected cross sections of the present device when extrapolated along it's flexed length, i.e. central (longitudinal) axis A is a flexible axis along the length of main tube 12.

It should also be understood, that the first end of the device 1, within the stomach may include alternative shaped ends to aid in initial insertion and assembly, or eliminate or prevent unintended withdrawal of the device through the patients stomach wall. For example, in an alternative embodiment to the smoothly rounded ends shown in present FIG. 3, a frusto-conical or enlarged bulbous head member (not shown) may be used. In this manner, when the to-be-described balloon is deflated, the device 1 hangs flaccid within the stomach with the enlarged head member drawn downwardly by gravity proximate the stomach inner wall, awaiting re-inflation.

Liquid chamber 6 includes a fluid chamber 6b extending between first and second sealed ends 10. 10′. A plurality of liquid ports 6a arrayed proximate the outer walls of liquid chamber 6, provide for a fluid communication between fluid chamber 6b and gastric balloon 2. In one preferred embodiment as shown, liquid chamber 6 projects outward from main tube 12 in a flexible form resistant to easy compression during use, but allowing reasonable compression and manipulation during insertion and removal.

It should be recognized that liquid chamber 6 is not restricted to the shape as shown, and may be any shape or size capable of receiving a medium of expansion (i.e fluid or fluid/gas combination) and distributing that medium of expansion effectively to balloon 2, while resisting detrimental compression (some compression is allowable absent functional detriment). Balloon 2 extends from about 10 cm to about 20-25 cm along the length of main tube 12 between sealed ends as shown. In this manner, balloon 2 may contain a large volume, expand without undue strain to fill a desirable portion of a patient's stomach, and distend as a normal stomach would under the forces of gravity. As a consequence of the present design and the size of balloon 2, the invention provides a substantially more effective and different clinical effect on a patient.

As will be described, during use balloon 2 may be filled with approximately 1500-1800 cc (or more or less depending upon patient size and other medical factors see below) of a suitable medium of expansion, frequently a liquid but allowing a combination of fluid and some gas, via ports 6a. As the medium of expansion passes through ports 6a, balloon 2 distends away from liquid chamber 6 and fill a patient's stomach, as will be described.

It should be understood by those skilled in the art, that the filed volume of balloon 2 is directed according to a variety of factors, including clinical need, patient conditions (including size, gastric health, physical structure, etc.), neuro-psychiatric conditions, and others. In sum, the volume used is determined within the bounds necessary for that patient as determined by a treating physician, and the above range should therefore be seen as an approximate and preferred range depending upon an average adult patient.

One or more pressure transfer channels 19A, 19A′, or 19A″ extends within or along the wall of main tube 12 from an opening proximate main end 17 to an opening on an outer wall of main tube 12 bounded by liquid chamber 6 in EPGB device 1. As shown, channel 19A opens into liquid chamber 6 and is in a fluid/gas communication with a separate opening at main end 17. It should be understood, that channel 19A is means for communicating at least one of hydraulic and a combination of hydraulic and bariatric pressure to an interior of chamber 6. Other means of communicating are envisioned and may include a passage separate from main tube 12 or a passage within passage 3. The present embodiments of channel 19A, 19A′, and 19A″ within main tube 12 (see generally FIG. 4) allows convenience, pressure resistance, patient safety, and ease of manufacture.

In use, a treating physician injects the medium of expansion into channel 19A under a pressure sufficient to resist an elastic urging of balloon 2 and causes balloon 2 to expand as necessary for treatment. In operation, liquid chamber 6 operates to receive the medium of expansion from channel 19A and distribute the medium of expansion along an outer diameter of main tube 12 and out ports 6A. In this manner, both the expansion medium and the expansion pressure it creates, are provided with an opportunity to reach a reasonable hydraulic area distribution so as to minimize sharp over-pressure in any one area and the related risk of blow-out or other failure mechanisms. In other words, the entire pressure of the medium of expansion is not directed to a very limited portion of balloon 2 or chamber 6, but is distributed over a wide area

A transgastric jejunal feeding tube 4, bounding and defining a feeding port passage 5 extends along a central opening 12a of main tube 12. Feeding tube 4 may be any type of commonly known feeding tube, some of which are made by Kimberly-Clark®, that allows simple feeding of the patient during treatment.

The difference between the outer diameter of feeding tube 4 and the inner diameter of central opening 12a creates a suction/wash lavage opening 3 or a port 3. As needed during use, gastric juices/waste fluids may be suctioned from a stomach (shown later in FIGS. 5, 5A) through passage 3, and washing/lavage/waste fluids may be injected through passage 3 to wash the stomach, as will be described. The presently shown preferred embodiment shows suction/wash lavage passage 3 surrounding the entire outer diameter of feed tube 4, but alternate embodiments or means to suction/wash/lavage are easily envisioned, for example a partial opening along or within the wall of main tube 12.

As will be noted later in FIGS. 5 and 5A, in a general embodiment of the present invention EPGB 1, feeding tube 4 further includes a weighted jejunal segment 4b at a tip end 15 to help maintain position (making treatment maintenance easier) and to ease installation of the device. In another preferred embodiment, the tip end 15 of feeding tube 4 may alternatively or additionally include a means to minimize clogging and improve fluid flow, such as a single feeding port (not shown) or multiple feeding ports 4a (shown).

As shown, an outer diameter of feeding tube 4 is less than an inner diameter of central opening 12a of main tube 12, thereby enabling feeding tube 4 to be easily inserted and withdrawn along central opening 12a, as needed as needed during use. In one preferred embodiment, an inflatable stop lock 14 (a stop means), proximate feeding tip end 15 of feeding tube 4 may be expanded during use to aid in securing tip end 15 along small intestine 22. Thus, inflatable stop lock 14 serves as a means to secure tip end 15 as needed in small intestine 22

Referring now to FIGS. 5, 5A, and 6, during use, one embodiment of an EPGB 1 is installed in a patient's body 13 using conventional installation techniques, resulting in main tube 12 and a main end 17 of EPGB 1 extending from a stoma or hub 9 at a surgically predetermined stoma cite 9a on a patients abdomen. EPGB 1 optionally includes feeding tube 4 having feeding end 15 opposite main or control end 17. During installation, feeding end 15 of feeding tube 4 is installed along a portion of small intestine 22, downstream of stomach 7, as shown.

The weighted jejunal segment 4b, (alternatively weighted end 4b), extending from feeding end 15 of feeding tube 4, aids the physician in placing feeding tube 4 in the medically correct position along small intestine 22.

In the preferred embodiment shown, once installed a treating physician operates end balloon stop lock 14 proximate feeding end 15 to removably secure feeding end 15 as desired in small intestine 22. Feeding part 4a, positioned proximate weighted end 4b and stop lock 14, allows the release of prepared food into a patient's small intestine 22 downstream from balloon 2 and stomach 7.

Feeding part 4a may be any type of conventional feeding part for a feeding tube 4. In the preferred embodiment shown, feeding part 4a includes multiple small exit ports allowing for a distributed food release and a minimized blocking. In another embodiment, feeding part 4a may be a singular large exit opening having an exit slot allowing easy release of any feeding materials and/or other nutrients as provided in the patient's feeding requirements.

To remove EPGB device 1, a physician releases stop lock 14, allowing feeding end 15 to slide freely from small intestine 22, and deflates balloon 2, similarly allowing the entire EPGB device 1 to be gently pulled through stoma 9 at stoma site 9a. The full insertion and removal method will be described in full detail below.

As noted in FIG. 6, main or control end 17 includes multiple ports allowing easy insertion, removal, and use of feeding tube 4 via feeding tube port 18. A first side liquid inflation port 19 is in a liquid communication with liquid chamber 6 along liquid channel 19A, and allows the ready inflation and deflation of balloon 2 in use. A second side suction/lavage/wash port 20, communicates with feeding tube port 18 and allows at least a liquid communication with a patient's stomach along suction/lavage passage 3.

Referring now to FIGS. 7 to 8, an alternative embodiment of the present invention is shown and described. A main tube 12′ extends, as generally described above and a pressure channel 19A′ extends generally parallel to a central passage 3′. A blockage 30 exists at an end of pressure channel 19A′ and blocks further downstream transfer of pressure. A balloon 2′ extends along a bottom portion of main tube 12′ along an outer wall of main tube 12′. Balloon 2′ is sealed at a first end proximate blockage 30 and at a second end upstream blockage 30. A central section of balloon 2′ is proximate an outer wall of main tube 12′ when in a contracted position, as shown.

A plurality of ports 6A′ communicate from pressure channel 19A′ to an inner surface of balloon 2′ between sealed ends of balloon 2′. Ports 6A′ may be formed in single or multiple rows, or in any configuration sufficient to provide a broad distribution of pressure along an inner surface of balloon 2′.

In use, a pressurizing medium, for example a liquid, is injected along pressure channel 19A′ and exits the plurality of ports 6A′ providing a broad pressure front to the inside surface of balloon 2′ thereby preventing pressure spikes, and allowing a large volume of pressurizing medium to be used without risk of breaking balloon 2′.

In an alternative embodiment of this present design, balloon 2′ may include an additional loose section (not shown) thereby containing additional material for expansion without maximizing volume in a deflated state.

It should be understood, that various solutions are available to distend and deflate the gastric balloon in the embodiments discussed. While we have discussed at least one way to distend and deflate the gastric balloon, others are readily available to those skilled in the art of designing medical devices and are intended to be incorporated herein. For example, the position of the pressure channel, use of multiple pressure channels, use of the main channel to deliver distending liquid are all available designs.

In contrast to FIG. 7, FIG. 8 discloses section of a conventionally known small air bladder having a main tube 12″ and a central passage 3″. A pressure channel 19A″ extends to a blockage 30″. A small balloon 2″ tightly wraps main tube 12″ and includes opposing sealed ends bounding a region containing a single cut opening 6A″ allowing air passage from pressure channel 19A″ to an inner surface of small balloon 2″. This conventionally known device is only effective to operate a small balloon 2″ and provides a very rigid expanded balloon, suitable for lodging the device in place. This is similar to the effects of stop lock 14, as noted above, namely to secure main tube 12″ in place by pressing against selected walls of a patient's digestive tract. Since this design is only effective to receive a gaseous pressure not a mass, the resulting expanded balloon 2″ is not effected by gravity, cannot expand and shift simulating the desired treatment basis and is consequently ineffective. Additionally, since cut opening 6A″ is a single opening, the risk of pressure spikes and material damage is increased, particularly to the walls of channel 19A″ proximate cut opening 6A″ and to the adjacent sections of balloon 2″.

II. Method of Treating Morbid Obesity Using an EPGB (Endoscopically Placed Gastric Balloon)

After carefully choosing the selective population, having a BMI over 40%, and further considering those with comorbid conditions a decision-discussion process was conducted to consider the risk-benefit ratio of the procedure and the alternatives if the procedure was not performed. Following this path, an endoscopically placed gastric balloon procedure was performed.

When reading the below discussion the following general phrases should be understood loosely. The discussion refers to percutaneous endoscopic gastrostomy/jejunostomy (PEG/PEJ), and devices commonly referred to as PEG and PEJ devices (gastrostomy or jejunostomy tubes). These devices are commonly known by those skilled in the medical arts. Additionally, the phrase lumen is employed loosely referring to an inner open space or cavity of a tubular organ, as of a blood vessel or an intestine, or more broadly used in the description to refer simply to an opening or passageway. Those skilled in the art, and understanding the highly skilled context, will understand the

The medical procedure for treating obesity or morbid obesity involving the present EPGB device 1 and alternatives involves multiple steps. After selection, the patient is placed preferentially in a left lateral position with a bite block, with mild sedation with propofol or other suitable sedation protocol. A video endoscope is passed through the bite block, over the tongue, and the pharyngeal recess and upper esophagus are visualized. Gastric distention is optimized to visualize up to the third part of the duodenum. The Anterior abdominal wall is exposed and optimal insertion is through the upper external abdominal wall. One-to-one movement was visualized and secured to the right position for insertion of a catheter from the sterile field from the upper abdominal wall.

2% Xylocane was given of local anesthesia on the sterile field. The trocar was introduced inside the stomach via the external abdominal wall. A snare was placed through the endoscope and caught the trocar at a snug and a guide wire was placed from the external abdominal wall inside the lumen via the trocar. The snare was released and placed carefully on the guide wire tightly and was pulled through the esophagus out of the mouth securing an access from the mouth through the esophagus and gastric lumen through the external abdominal wall outside. A prototype PEG was attached through that guide wire and gently pulled via the mouth, esophagus, and the stomach. A minor incision was given on the external abdominal wall to facilitate the prototype balloon PEG to pull out of the external abdominal wall keeping the balloon and the PEG inside the gastric lumen.

A PEJ was introduced via the PEG from the external side, which was visualized in the stomach lumen by endoscope. An alligator tooth forceps was introduced via gastroscope and was placed on the PEJ and eventually pushed that PEJ beyond the ligament. The alligator forceps was expelled out of the endoscope. The endoscope was used to visualize with accuracy the position of the PEJ in the part of jejunum. The endoscope was removed up the lumen and out of the mouth. Hence one prototype gastric balloon PEG was placed in the gastric lumen.

Out of three ports external, one part was used to inflate the balloon with 1800 cc of normal water or saline. The balloon will expand or does expand upon inflation to occupy the entire lumen including the GE junction. The esophageal orifice is also obliterated for larger solid or even semisolid food, excepting passage allowing saliva to dribble out via the esophageal orifice into the gastric area.

A second port is used for the PEG during feedings at designated antral feedings, at night via a pump while the patient is sleeping. Another port is used for suction and lavage of the gastric lumen and placement of soluble PPI (lansoprazole 30 mg to prevent acid reflux), thus minimizing corrosion and erosion of the balloon and organic surfaces. These steps are necessary for maintaining and manipulating the gastric nexus into a steady state.

Gastric lavage was preformed to keep the lumen clean from bile, pepsin, etc. the Gastric balloon port is used periodically to deflate the balloon and to inflate it again with fresh water or normal saline. The port out of the external abdominal wall should have a stump to modify it for aesthetic purposes and for the area to remain clean and avoid infection and leakage of gastric juice material.

The proposed balloon would be kept by the patient until the derived weightless, which should be achieved slowly and progressively to avoid Nonalcoholic Steatohepatitis (NASH), fatty liver decomposition, and other complications as precipitation of gallstones, etc. Optimal balloon stay is presumed 6-8 months.

After the weight loss is achieved, a deflated balloon and the PEG may be removed through the external abdominal wall and the orifice of the PEG is healed with natural closure. This type of removal is an extremely simple procedure and has been used over the years for antral feeding, or with people with terminal release, in nursing homes, with laryngeal or pharyngeal cancer, etc.

Complications of this procedure are estimated to be similar to those of PEG, namely infection of the PEG site, rupture of the balloon, bleeding at the site, and very rarely displacement of the balloon. Aspiration pneumonia, particularly saliva or gastric juice are much less common than previously existing PEG pegged situations as the feeding material is placed in the jejunum avoiding the stomach, and due to the periodic lavage (within 24 hours) of gastric juice and saliva.

After review of the above, it should be understood by those skilled in the art, that the balloon contents of 1800 cc inflated, within the gastric lumen, provides satiety and manipulation of neurogastric hormonal receptors aiding the patient's weight loss. As a result of one or more of the device, the lavage practice, and the use of the present method and device to manipulation of the neurogastric hormonal receptors, patients may be treated to combat morbid obesity.

In sum, the present reversible treatment solution consists, in part, of endoscopic removable placement of EPGB device 1 that is temporarily inflated/distended with at least a liquid medium (preferably sterile or normal water, but may include another liquid or a small portion of gas) up to about 1800 cc depending on patient size and other medical considerations.

In this manner, the treatment keeps the gastric inlet distended and this results in a bio-generation and communication of a satiated state without the use of actual food consumption by mouth, thereby both altering the intrinsic neurogastric hormonal repertoire and equally delivering restricted nutritional support destined to sustain progressive weight loss while avoiding the metabolic complications noted earlier.

It should be understood by those skilled in the art that, as used herein, the phrase ‘enteral’ or ‘enteral feeding’ or other similar phrase refers to the intestine or gastrointestinal tract. See Stedman's Medical Dictionary 27^{th} Ed (2003). It should be similarly understood, that the phrase fill or full, or the precise balloon volume employed should be reasonably determined based upon the patient's condition as viewed by a treating physician (See FIG. 1 suggesting the broad range of weight and heights considered). For example, treating an obese woman standing 4′ 9″ would likely suggest a different volume/full amount, than treating a 6′ 10″ man. Consequently, the volumes and the use of the phrases fill or full are to be understood as relative to a particular patient and a particular treatment regime modified for that patient.

It should be additionally understood by those skilled in the art that, as used herein, the phrase ‘bariatric’ or ‘bariatric surgery’ or other similar phrases refers to a branch of medicine that deals with the causes, management, prevention, and treatment of obesity and allied diseases. See www.Dictionary.com, (Jul. 28, 2003) and Dept. of Medical Oncology, University of Newcastle upon Tyne, UK.

It should be additionally understood by those skilled in the art, that the present EPGB device does not require feeding tube 4 to operate and provide a satiated feeling to a patient, the feeding tube is provided for sustaining the patient during treatment. It will be recognized, that in alternative embodiments and methods, feeding tube 4 may be removed and replaced for various reasons, i.e. prevent infection, minimize blockage. Consequently, while the phrase EPGB device has been used to refer to the entire present invention 1 (including feeding tube 4), the EPGB device may be used and referred to without feeding tube 4, as the walls of main tube 12 are sufficiently stiff to withstand detrimental pressure from balloon 2 during inflation and use.

It should be understood, that there are many known means to seal and secure two plastic elements together as in the present invention, these methods include ultrasonically welding or securing, the use of retaining bands or adhesive or any other known means for securing two elements together in a manner suitable for use in a surgical environment.

In the claims, means- or step-plus-function clauses are intended to cover the structures described or suggested herein as performing the recited function and not only structural equivalents but also equivalent structures. Thus, in a simplified example, although a nail, a screw, and a bolt may not be structural equivalents in that a nail relies on friction between a wooden part and it's cylindrical outer surface, a screw's helical surface positively engages the wooden part, and a bolt's head and nut compress opposite sides of a wooden part, in the environment of fastening wooden parts, a nail, a screw, and a bolt may be readily understood by those skilled in the art as equivalent structures.

Having described at least one of the preferred embodiments of the present invention with reference to the accompanying drawings, it is to be understood that the invention is not limited to those precise embodiments, and that various changes, modifications, and adaptations may be effected therein by one skilled in the art without departing from the scope or spirit of the invention as defined in the appended claims.

What is claimed is:

1. An endoscopically placed gastric balloon (EPGB) device, comprising
a main tube having a defined central region and at least a first end opposite a second end;
at least a first liquid chamber bounding a portion of an outer diameter of said main tube for receiving and transmitting a preselected fluid medium;
at least a first balloon member proximate an outer diameter of said first liquid chamber;
means for communicating a fluid pressure between said at least liquid chamber and said balloon member;
means for enabling an inflation of sad first balloon member including means for enabling an injection of a preselected fluid into said liquid chamber, through said means for communicating and into said first balloon, thereby communicating said fluid pressure and distending said balloon during a use of said device; and
said means for enabling including a plurality of radially arrayed ports.

2. An endoscopically placed gastric balloon (EPGB) device, comprising:
a main tube having walls bounding at least feeding and lavage passage therethrough;
a gastric balloon member bounding at least a first portion of said main tube and defining an elastic liquid chamber thereabout;
means for providing a controllable fluid communication to said liquid chamber; and
said means for providing including at least one fluid pressure channel in said walls for transmitting said fluid and at least one port member accessing said liquid chamber, whereby fluid is communicated to said liquid chamber for inflation and deflation during a use.

3. An endoscopically placed gastric balloon (EPGB) device, according to claim 2, further comprising:
means for removably securing said gastric balloon member and at least a portion of said main tube within an external patient's stomach.

4. An endoscopically placed gastric balloon (EPGB) device, according to claim 3, further comprising:
a transgastric jejunal feeding tub member; and
a means for securely positioning said feeding tube member within an external patient's intestinal tract.

5. An endoscopically placed gastric balloon (EPGB) device, according to claim 3, further comprising:
means for enabling a lavage of said patient's stomach, whereby during said use a patient stomach may be cleaned without removing said EPGB device.

6. An endoscopically placed gastric balloon (EPGB) device, according to claim 2, further comprising:
a plurality of fluid communication ports in said means for providing, whereby said plurality of ports enables a rapid inflation and deflation of said gastric balloon member during said use.

7. A method for treating morbid obesity, comprising the steps of:
surgically positioning an endoscopically placed gastric balloon (EPGB) device in a suitable patient, said EPGB comprising:
a main tube having a defined central passage and at least a first end opposite a second end;
at least a first liquid receiving chamber sealed to an outer diameter of said main tube for receiving at least one of a fluid and a gas and a fluid combination;
means for communicating a fluid pressure to said liquid receiving chamber and a gastric balloon member bounding said liquid receiving chamber during a use; and
means for controllably injecting at least a preselected fluid into said liquid chamber, through said means for communicating, and for distending said balloon during said use, thereby causing a patient to feel a satiated hunger;
inflating said device during said use, whereby said patient feels a satiated hunger craving;
deflating said gastric balloon member at periodic intervals;
operating said device to remove, lavage, and suction detrimental fluids from said patient's stomach at selected times;
inserting a transgastric jejunal feeding tube along said central passage during one of said steps of inflating and deflating;
feeding said patient via said feeding tube member during one of said steps of inflating and deflating, whereby the nutritional requirements of said patient are maintained in a convenient manner, while maintaining said satiated hunger craving status.

8. A method for treating morbid obesity, according to claim 7 further comprising the step of:
injecting substantially 1800 cc of fluid into said gastric balloon member during said step of inflating.

9. A method for treating morbid obesity, according to claim 8 further comprising the step of:
securing said feeding tube in said patient prior to said step of feeding, whereby said method improves a safety of treating morbid obesity.
